(19) United States
(12) Patent Application Publication
(10) Pub. No.: US 2011/0301461 A1
(43) Pub. Date: Dec. 8, 2011

(54) SELF-ADMINISTERED BREAST ULTRASONIC IMAGING SYSTEMS

(76) Inventor: Doris Nkiruka Anite, Victoria Island (NG)

(21) Appl. No.: 12/857,404

(22) Filed: Aug. 16, 2010

Related U.S. Application Data
(63) Continuation-in-part of application No. 12/826,188, filed on Jun. 29, 2010.
(60) Provisional application No. 61/357,953, filed on Jun. 23, 2010.

(30) Foreign Application Priority Data
Jun. 4, 2010 (NG) NG/P/2010/374

(51) Int. Cl. A61B 8/14

(52) U.S. Cl. 600/443

(57) ABSTRACT

An ultrasonic breast examination system suitable for use by unskilled users, comprising a patient positioning platform with a patient positioning image sensor, and a moveable ultrasonic transducer guidance device. The transducer guidance device can be a fluid filled container with a flexible side or bottom, and a mechanism to automatically move an ultrasonic transducer over the breast. The system will be controlled by at least one microprocessor, associated software, and an optional touch sensitive display screen. The system may use an image sensor to properly position the patient so that the transducer guidance device may be properly positioned proximate to the patient's breast. The touch sensitive display screen is designed to allow the system to be directly operated by an unskilled patient. The system will often be connected to a network, such as the Internet, so that remote operators may interpret breast ultrasound images and optionally control the system.

### CROSS REFERENCE TO RELATED APPLICATIONS

This application is a continuation in part of U.S. patent application Ser. No. 12/826,188, “Self-administered Medical Ultrasonic Imaging systems, filed on Jun. 29, 2010, Dr. Doris Nkiruka Anite inventor; U.S. application Ser. No. 12/826,188 in turn claims the priority benefit of Nigerian patent application NG/P/2010/374, entitled “SELF-ADMINISTERED DIAGNOSTIC MEDICAL DEVICES AND SOFTWARE”, filed on Jun. 4, 2010, and U.S. Provisional Application No. 61/357,953, filed on Jun. 23, 2010, also entitled “SELF-ADMINISTERED DIAGNOSTIC MEDICAL DEVICES AND SOFTWARE”, both by Dr. Doris Nkiruka Anite inventor.

### BACKGROUND OF THE INVENTION

1. Field of the Invention

The application is in the general field of medical devices and medical diagnostics, particularly ultrasonic imaging technology.

2. Description of the Related Art

Medical imaging technology, in particular ultrasonic medical imaging technology, has revolutionized the diagnosis and care of many medical problems, such as screening for breast cancer, complications of pregnancy, monitoring the status of various pelvic and abdominal organs, monitoring cardiac function, and many other areas as well. Ultrasonic medical imaging technology is widely used because unlike other forms of medical imaging technology, such as X-rays, ultrasonic scans are better able to detect soft tissue problems and are also generally considered to be quite safe. Ultrasonic imaging equipment, although certainly not inexpensive, also contrasts favorably in expense to other much more expensive forms of safe medical imaging technology, such as magnetic resonance imaging (MRI) methods. Medical ultrasonic imaging is often referred to in the alternative as medical ultrasonography, diagnostic sonography, and the like.

Ultrasonic imaging technology essentially works by a sonar-like echo method. An ultrasonic transducer emits focused beams of ultrasonic energy, typically in the form of very high frequency vibrations in the 2 to 18 megahertz range. This is far above the normal range of human hearing, which generally extends no further than about 20 kilohertz, hence the name ultrasonic.

Ultrasonic imaging methods work well for imaging soft tissues, as the sound waves tend to reflect off of the interface between different soft tissues, creating an echo pattern that can be analyzed with the use of computerized equipment and translated into a visual image of the various tissues that underlie the ultrasonic transducer. The further away a particular tissue junction is from the transducer, the longer the sound wave pulse will take to travel from the transducer head to the tissue junction, and then back again to the transducer head, and this time lag thus can be used to determine the distance to that particular soft-tissue junction.

One problem with ultrasonic imaging technology, however is that this technology still requires that a skilled operator be present to move the ultrasonic transducer about the body, and interpret the images on the video monitor of the computerized ultrasonic imaging equipment. This need for a skilled operator is unfortunate, because due to advances in electronics and instrumentation, the basic costs of medical ultrasonic imaging equipment itself is likely to continue to substantially decrease over the coming years. However since skilled operators are scarce, these equipment cost improvements may not translate into broader access to this technology. Moreover, such skilled operators or workers are often not available or on call in remote locations, or outside of standard business hours, when many medical emergencies occur.

As a result, patients suffering from sudden medical problems in remote locations, or outside of standard business hours, may be denied the substantial benefits of rapid medical diagnosis that ultrasonic imaging technology might otherwise offer. As a result, the present situation is unsatisfactory.

Breast cancer is a major disease, but due to the inconvenience of conventional imaging technology, even women who may be concerned because they feel suspicious breast lumps may be discouraged from undergoing screening until it is too late. Thus improved methods of obtaining diagnostic medical images, in particular ultrasonic medical images of the breast, without the immediate availability of trained operators, would be of high medical utility.

### BRIEF SUMMARY OF THE INVENTION

What is needed is an automated or semi-automated device and method that can be used by local unskilled users or anyone to produce good quality medical ultrasonic images of the breast or other body portions of interest. These images in turn could then either be transmitted to a remote skilled interpreter of diagnostic images, such as a remote physician or technician, by way of a conventional network such as a cell phone network, land line phone network, the Internet, radio linkages, or other telecommunications means. The remote skilled image interpreter could then examine the images and make recommendations as to what subsequent medical steps should be done.

Alternatively, even in the absence of a network link to a remote skilled interpreter, an automated or semi-automated device and method used to produce skilled medical images could still be of high utility even when used in a mode where a patient might have a scan done in a remote location or after hours setting, and the medical image either printed out or saved in a computer memory storage device for subsequent analysis by a skilled interpreter. This way the patient could, for example, come in, get a scan, and report back to a physician only if further intervention was necessary, thus saving unnecessary travel and strain on the patient.

If such a device and/or method existed, then patients would be more likely to monitor themselves more often, check up on suspicious lumps or other breast abnormalities, and thus breast diseases could be caught and treated at a much earlier stage, when survival rates are much higher.

In principle, ultrasonic medical imaging is well suited for automation and simplification or “de-skilling”. This is because, as previously discussed, the ultrasonic vibrations produced by ultrasonic transducers are generally considered to be safe. Further, with the exception that occasionally a water based gel is placed between the ultrasonic transducer and the patient's skin in order to improve the coupling between the ultrasonic transducer and the patient's skin, the process of moving the ultrasonic transducer about different regions of the patients body is not particularly complex.

Thus, according to the invention an improved device and method for producing medical diagnostic ultrasound images of body portions (such as the breast), suitable for unskilled users, may be obtained by constructing a device that de-skills the process of positioning the ultrasonic transducer on the patient's breast, and subsequently moving the ultrasonic transducer about the different regions of the patient's breast. Ideally this device would also be coupled with a network based communications device that relays the ultrasonic medical images to a remote user, who may be a skilled interpreter of medical images. Alternatively or additionally, a local image storage device such as a printer or computer storage that enables the ultrasonic medical images to be read at a later time may also be coupled with the improved de-skilled ultrasonic scanner device.

For breast scans, which require relatively small and precise movements in order to capture potentially small lumps and other suspicious regions, the preferred way to de-skill the process is to automate the process. This can be done by means of one or more actuators (e.g. electric motors and the like) that move the ultrasonic transducer head in the proper manner under the control of a computer processor and software, or by a remote skilled operator, or a combination of both.

The process of de-skilling or making the medical ultrasonic scanning process simpler may generally involve use of sensors to properly orient the patient and the patient's breast, as well as determine the relative position of the ultrasonic transducer on the patient's body. The process may also generally require a guidance device or platform to help guide the motion of the transducer. Additionally, computational means such as at least one microprocessor, software to read the sensors and to direct the motion of the transducer may generally also be used. The system may also make use of the equipment normally associated with the generation and production of ultrasonic images, such as acoustic transducers, acoustic receivers, computer based processors, and the like. In principle all modes of sonography, including A-mode, B-mode, M-mode, and the various Doppler modes may run on the simplified ultrasonic guiding systems according to the invention.

Thus, in one embodiment, the invention may be viewed as a medical ultrasonic transducer guiding system capable of being used by unskilled users. This system will generally comprise at least a moveable guidance platform configured to enable an ultrasonic transducer to be repositioned in multiple positions about a patient's breast. This guidance platform may also have sensors that detect the position of the patient's breast, as well as the position of the ultrasonic transducer, and produce output describing the position of the ultrasonic transducer. This transducer position sensor data will usually be processed by at least one processor (often a computer microprocessor) and associated positioning software that receives the transducer position output from the transducer location sensors. This software will direct the position of the ultrasonic transducer in a manner that may depend upon a preset algorithm, or depend on directions from a remote operator. The system will usually also have electronic circuitry to drive the ultrasonic transducer, as well as circuitry to receive the ultrasonic echo information back from the transducer. The system will additionally have image reconstruction software to take this echo information and assemble one or more images showing the interior of the patient's body.

Thus, during the course of an automated scanning session, the positioning software will direct the ultrasonic transducer to be repositioned in multiple locations about a patient's breast, and the said image reconstruction software will assemble at least one image representing the interior of said patient's body. This image will be viewed by a remote skilled user, for example over a network (e.g. the Internet), or on a time-shifted basis by a local skilled user at a later time.

Thus in some embodiments, the invention is a system and method enabling unskilled users to perform ultrasonic breast examinations. The system will often be made up of a patient positioning platform with a patient positioning image sensor, and a moveable ultrasonic transducer guidance device. The transducer guidance device can be a fluid filled container with a flexible side or bottom, and will usually also contain one or more mechanisms to automatically move at least one ultrasonic transducer over the patient's breast or breasts. The system will usually be controlled by at least one microprocessor, associated software, and optionally a touch sensitive display screen. The system may optionally use an image sensor, along with suitable guidance software, reference marks, and instructions, to properly position the patient so that the transducer guidance device may be properly positioned on top of the patient's breast. The touch sensitive display screen may be designed to allow the system to be directly operated by an unskilled patient without the need of any additional local help. The system will often be connected to a network, such as the Internet, so that remote skilled users, such as physicians and skilled technicians, may interpret breast ultrasound images and optionally control the system.

Although, throughout this specification, the example of a breast is used as a specific example of one application of this system and method, it should be evident that this system and method may be used to image other portions of a patient's body as well.

### BRIEF DESCRIPTION OF THE DRAWINGS

FIG. 1 shows one embodiment of the ultrasonic breast examination system with the moveable ultrasonic transducer guidance device positioned in the patient positioning platform above the patient's breast. In this configuration, the patient is laying down.

FIG. 2 shows the ultrasonic breast examination system with the moveable ultrasonic transducer guidance device positioned proximate to the patient's breast. In this configuration, the patient is laying down.

FIG. 3 shows a top down view of the moveable ultrasonic transducer guidance device positioned above a patient's breast, showing some of the components of the device.

FIG. 4 shows an internal side view of the moveable ultrasonic transducer guidance device, in this view positioned on top of (proximate) to the patient's breast.

FIG. 5 shows an alternate embodiment of the device, more closely resembling a traditional mammography scanner, in which the patient is using the device standing up.

FIG. 6 shows an alternate embodiment of the ultrasonic breast examination system with the moveable ultrasonic transducer guidance device (114) positioned to be adjusted up and down in height relative to the base (100) to match various patient breast heights. In this configuration, the patient is standing up.

FIG. 7 shows an example of an image of the patient's breasts as displayed on the display device, showing how the system processor and software can display commands to direct the patient to manually reposition her body on the patient positioning platform.

FIG. 8 shows an example of how a breast positioning image sensor inside the moveable ultrasonic transducer guidance device (114) may look at the patient's breast through a transparent flexible side or bottom once the ultrasonic transducer guidance device is positioned (proximate) to the patient's breast. The image sensor can then produce images of the patient's breast, which may be also displayed on the display device, and used both to insure that the breast is properly positioned prior to scanning, and also can be used to help control the breast scanning process

FIG. 9 shows an example of how either a patient or other system operator may interact with a touch screen display device and, for example, inform the system as to the position of the boundaries of the breast as it is positioned underneath the ultrasonic transducer guidance device. Alternatively the positions of the breast boundaries can be determined by artificial vision systems or other user input modalities.

FIG. 10 shows an example of how either a patient or other system operator may interact with a touch screen display device and, for example, inform the system as to the position of the areola region of the breast as it is positioned proximate to the ultrasonic transducer guidance device. Alternatively positions of the breast areola or other breast structures can be determined by artificial vision systems or other user input modalities.

FIG. 11 shows an example of what the display device, which may be a touch screen display device, may look like during a breast scan process. This type of interface allows the patient to see the progress of the scan, and also allows the patient to abort the scan early if the patient feels uncomfortable.

FIG. 12 shows an example of system being controlled over a network, such as the internet, by a remote user. In this example, the laying down version of the device is shown.

FIG. 13 shows an example of the electronics and software used in the system.

### DETAILED DESCRIPTION OF THE INVENTION

Here ultrasonic transducers will occasionally be referred to in the alternative as the ultrasonic probe, ultrasonic probe head, or occasionally the probe head or probe.

As previously discussed, the invention may be viewed as a medical ultrasonic transducer guiding system capable of being used by unskilled users. This system will generally comprise at least a moveable ultrasonic transducer guidance platform configured to enable an ultrasonic transducer to be repositioned in multiple positions about a patient's breast (either one breast at a time, or both breasts at a time), and produce useful medical diagnostic images of the patient's breast.

This guidance platform may optionally also have or comprise sensors that detect the position of the patient, the patient's breast, and the ultrasonic transducer. These sensors may include image sensors, as well as ultrasonic transducer position sensors that may be built into the actuators that move the ultrasonic transducer, or may be external to the actuators. This sensor data will often be processed by at least one processor (often a computer microprocessor) and associated positioning software that receives the output from these sensors and directs the position of the ultrasonic transducer depending upon a preset algorithm, or upon further direction from a remote operator. The system will usually also have electronic circuitry to drive the ultrasonic transducer, as well as circuitry to receive the ultrasonic echo information back from the transducer. The system will additionally have image reconstruction software to take this echo information and assemble one or more images showing the interior of the patient's body. This software will generally work in conjunction with other software and electronic systems, such as conversion software to make it easier to transmit images over a network, network software, network routers, communications links, interfaces, operating systems, and the like.

During the course of an automated scanning session, the positioning software will direct the ultrasonic transducer to be repositioned in multiple locations about the patient's breast, and this image reconstruction software will assemble at least one image representing the interior of the patient's breast. This positioning software will normally direct the ultrasonic transducer to move by issuing movement command to various mechanical actuators, such as motors, solenoids, pistons, and the like, or by issuing movement commands in an audible or written form intended to be implemented by a human user, such as the patient or operator of the system. In this later case, a processor that controls the guidance platform or other processor(s) may be hooked up to an audio output device, such as a speaker, and issue verbal commands. Alternatively the processor may be hooked up to a video display and issue written commands and/or show diagrams instructing the user as to how to use the device. Alternatively a mixture of machine transducer and human movement commands may be issued.

This moveable ultrasonic transducer guidance device will normally be affixed to a patient positioning platform that is designed to insure that the moveable ultrasonic transducer guidance device is positioned correctly relative to the patient's breast. Usually the patient will be a human patient but in some cases the patient may be an animal patient, and the device will be used for veterinary purposes.

As previously discussed, in many of the embodiments, it may be useful to connect the device to a network, such as a cell phone network, land line phone network, the internet, or other type of network so that the images produced by the ultrasonic scanner can be viewed by a remote user/operator, who may be a skilled operator such as a physician or technician. In other embodiments, this remote operator may also receive ultrasonic transducer location information over the network from the guidance platform, and this remote operator may in turn either transmit instructions to the processor running the guidance platform to operate mechanical actuators to move the transducer to a desired location on the patient's breast, transmit instructions to change the parameters of the ultrasonic scan, transmit verbal instructions to local users, or some combination of the above.

Thus in one embodiment, the invention may be a medical ultrasonic breast examination system for a patient. In this embodiment, the invention may comprise a patient positioning platform with an optional patient positioning image sensor and, a moveable ultrasonic transducer guidance device. This moveable ultrasonic transducer guidance device in turn may be a container (which often may be a fluid filled container), such as a box with a flexible side or bottom. Inside the moveable ultrasonic transducer guidance device, there will generally be both at least one moveable ultrasonic transducer as well as mechanisms to automatically move this ultrasonic transducer, at least within the confines of the container. The flexible side or bottom will often be a membrane, such as a fluid impermeable membrane, capable of conforming to the shape of at least one patient's breast when the device is positioned about this breast or breasts. The membrane may optionally be an elastic membrane so as to better conform to the shape of the patient's beasts without wrinkling. The transducer guidance device will often be held in a patient positioning platform by mechanisms that enable the device to move from at least a first position away from the patient's breast, to a second position proximate (immediate contact with) the patient's breast. For example, in embodiments in which the patient is laying down, then the transducer guidance device may move from a position above the patient's breast(s) onto the patient's breast(s). In embodiments in which the patient is standing up (or sitting), then the transducer guidance device may have an adjustable height so as to match the patient's height and breast position.

The system may optionally have at least one display device, such as a touch sensitive video display screen, configured to display at least some of the images from the optional patient positioning image sensor in order to allow the patent's breast to be properly aligned in the system. The system will also generally have at least one processor (such as a microprocessor) configured to automatically move the transducer in a scanning pattern intended to enable echo information obtained by the ultrasonic transducer to be assembled into at least one image representing the interior of said patient's breast.

In an alternative embodiment, the invention may be a method for automatically performing a medical ultrasonic breast exam of a patient. In this embodiment, the method comprises obtaining a patient positioning platform with at least one patient positioning image sensor, and, a moveable ultrasonic transducer guidance device. This moveable ultrasonic transducer guidance device may, for example, comprise a fluid filled container with a flexible sides or bottom, a moveable ultrasonic transducer configured to move within said container, and a mechanism to automatically move this moveable ultrasonic transducer about at least one patient's breast. The method may optionally involve using at least one patient positioning image sensor to obtain patient positioning data to instruct the patient to position herself in the proper position on the patient positioning platform to perform an ultrasonic breast exam her breast. The method also involves positioning this moveable ultrasonic transducer guidance device so that the device's flexible sides or bottom conforms to the shape of the patient's breast, and the pressure of the fluid on the flexible side or bottom causes the flexible side or bottom to tightly adhere at least one of the patient's breasts. The method also involves using a processor and software to direct the mechanism to automatically move the ultrasonic transducer about the patient breast or breasts, thereby obtaining ultrasonic image data. This ultrasonic image data can then be transformed or assembled into at least one image representing the interior of the patient's breast. This image may be transmitted over the internet and interpreted by a remote user. Alternatively a remote operator may control the breast scan over the internet by sending commands to the system's processor and software.

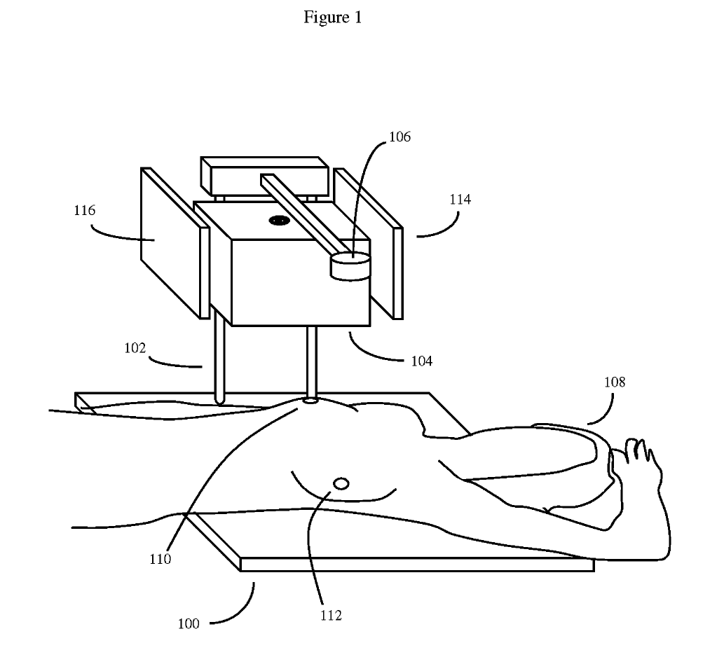

FIG. 1 shows the ultrasonic breast examination system with the moveable ultrasonic transducer guidance device positioned in the patient positioning platform above the patient's breast. In this embodiment the patient is laying down.

In this embodiment, the system has a patient positioning platform (100) which contains structural elements (such as metal poles) (102) capable of supporting a moveable ultrasonic transducer guidance device (104) and an optional at least one patient positioning image sensor (106) which may be a video camera and/or a light source and a video camera. In some embodiments, the video camera may optionally be a multi-spectral video camera capable of photographing the patient (108) and the patient's breasts (110), (112) in multiple wavelengths, including standard visible light wavelengths (e.g. red, green, blue) and also other wavelengths such as infrared wavelengths.

In some embodiments, the ultrasonic transducer guidance device (104) may be designed to cover and analyze only one of the patient's breasts at a time. This configuration may be less costly, and may, at least in the laying down embodiment, also be more tolerated by claustrophobic patients because since one side of the patient's body is always uncovered, the patient is thus free to wiggle out of the device at any time and terminate the scan. In this laying down one breast at a time embodiment, the patient (108) may first position herself on the patient positioning platform with her head in a first direction, and have the first breast (110) scanned. The patient may then get up, position herself on the patient positioning platform with her head in the opposite direction, and then get her other breast scanned (112). Alternative versions of the device that scan both breasts at the same time may also be used.

In some lower cost embodiments, the position of the ultrasonic transducer guidance device (104) on the patient positioning platform (100), (102) may be moved, (e.g. moved up and down) manually by the patient or the operator, and the ultrasonic transducer device held in an up position by catch mechanism or other type of mechanism, and may be held in the down position merely by the weight of the device (104) which often will have a weight between about 10 and 40 pounds, which is sufficient to hold the device (104) firmly against the breast (110), but not so firmly that the patient cannot manually push the device away and exit the platform (110) if the patient so desires. This “fail safe” mechanism may be particularly good for situations where the patient may be expected to use the system without any additional local help.

In more premium embodiments, the position of the ultrasonic transducer guidance device (104) may be controlled by an automatic mechanism, such as an electric motor, and often under control from a user input device such as a touch sensitive display screen (114) or other mechanism.

In order to help operate the device, the device may optionally contain at least one display device, such as a video screen or touch sensitive video screen. In some embodiments, this display device may be positioned so that the patient herself may easily view the display device and interact with the display device, even during the course of a breast scan. In the embodiment shown in FIG. 1, the display device (114) is shown attached to the side of the ultrasonic transducer guidance device (104). In order to facilitate use when the patient's head is positioned in the opposite direction, a second display device (116) is also shown mounted to the opposite of the ultrasonic transducer guidance device (104). It should be appreciated, however, that in fact the display device or display devices may be positioned in a variety of different locations, either attached to the patient positioning platform (110) or not.

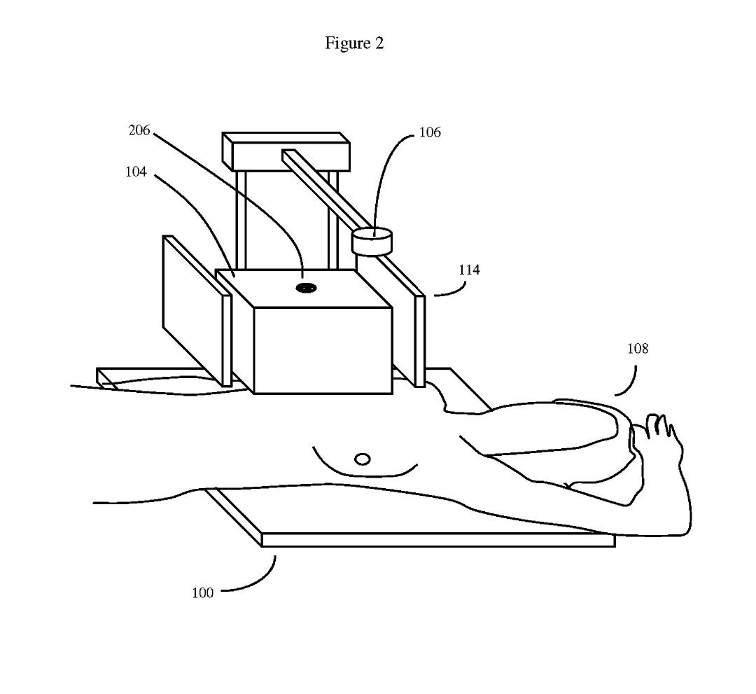

FIG. 2 shows the ultrasonic breast examination system with the moveable ultrasonic transducer guidance device positioned proximate to the patient's breast. In this position, the patient is still free to view the progress of the test on display device (114) and if display device (114) is a touch sensitive display device, the patient may also answer questions, control the scan, and terminate the scan early if the patient so desires.

In some configurations, the ultrasonic transducer guidance device (104) may have a transparent flexible side or bottom, as well as at least one breast positioning image sensor (206) capable of photographing the patient's breast (110) or breasts that are positioned underneath the ultrasonic transducer guidance device (104) when this device is lowered or otherwise moved into a position proximate the patient's breast. This breast positioning sensor (206) can be very useful because often the patient's breast (110) will deform or change position slightly in response to pressure from the weight of the sides or bottom of the ultrasonic transducer guidance device (104), and sensor (206) may thus provide data to both ensure that the breast is properly scanned, as well as provide data useful for determining the relative position and boundaries of various breast structures have shifted during the scan.

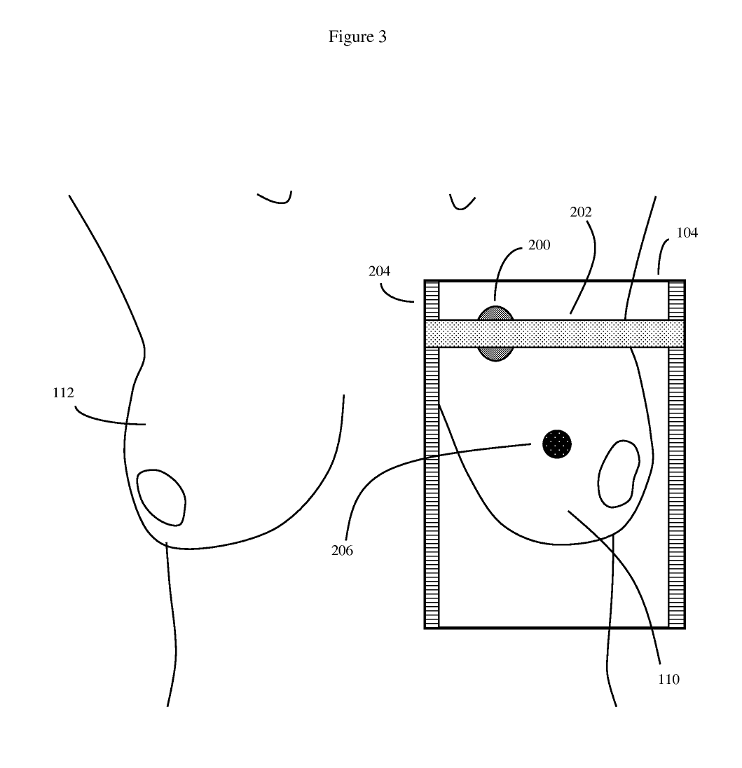

FIG. 3 shows a top down view of the moveable ultrasonic transducer guidance device (104) positioned above a patient's breast (110), showing some of the components of the device. In addition to an optional breast positioning image sensor (206), device (104) will generally contain an ultrasonic transducer (200) suitable for producing ultrasonic images of a portion of the breast, as well as positioning mechanisms (202), (204) and actuators designed to move the ultrasonic transducer (200) within the confine of the guidance device (104). Often this movement will be controlled by one or more processor and software controlled electric motors (e.g. actuators) (not shown). For simplicity, here some of the other components of the system, such as the patient positioning platform, display devices, patient positioning image sensor, and the like, are not shown.

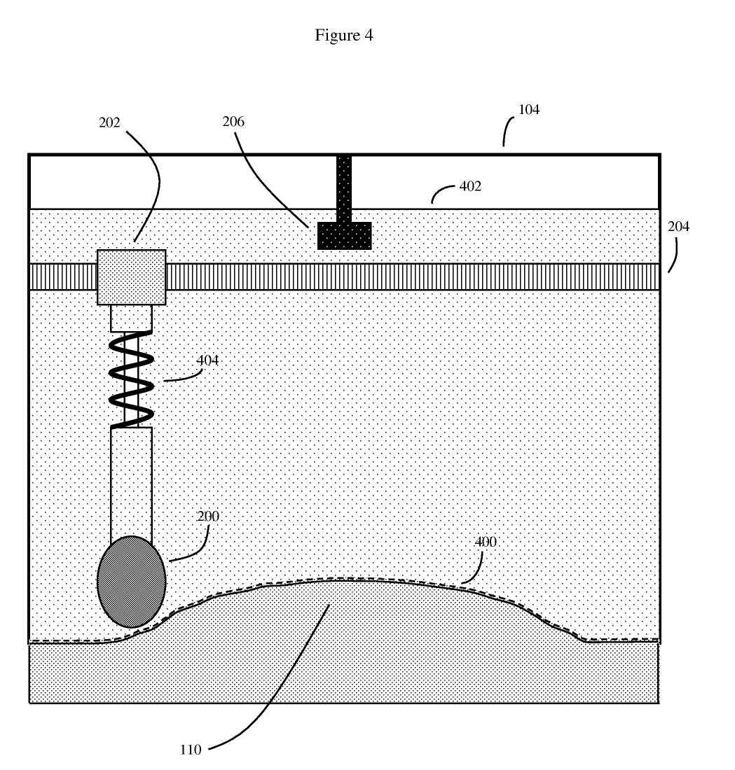

FIG. 4 shows an internal side view of the moveable ultrasonic transducer guidance device (104), in this view positioned on top of (proximate) to the patient's breast (110). Often it will be useful to make the bottom of the device out of a flexible membrane (400) such as plastic, that is both capable of adhering tightly to the outlines of the patient's breast (110) (which in some embodiments may be covered with a layer of water gel prior to the scan) and also capable of transmitting light so that image sensor (as well as optional light source (206) may image the patient's breast directly underneath device (104). The interior of the device (104) may optionally be filled with a liquid material, such as water or silicon oil (402), so that membrane (400) is under constant pressure to adhere firmly to the breast (110) throughout the scan. This hydrostatic pressure can also help keep the breast reasonably stationary throughout the exam. The ultrasonic transducer (200) may additionally be held by a mechanism (here a simple spring mechanism is shown) (404) that causes the ultrasonic transducer to press on the breast with a relatively constant pressure as it is moved about the surface of the breast by mechanisms (202) and (204).

To further insure that there are no air pockets between ultrasonic transducer head (200) and the patient's skin, a layer of a water soluble gel (not shown), may be used. In one embodiment, this gel may be provided in a pre-packaged container designed for easy application by a patient. The gel may be applied on top of the patient's breast before the scan. Once device (104) is in position, the gel may form a thin layer between the underside of the thin flexible membrane (400) and the patient's breast (110, 112).

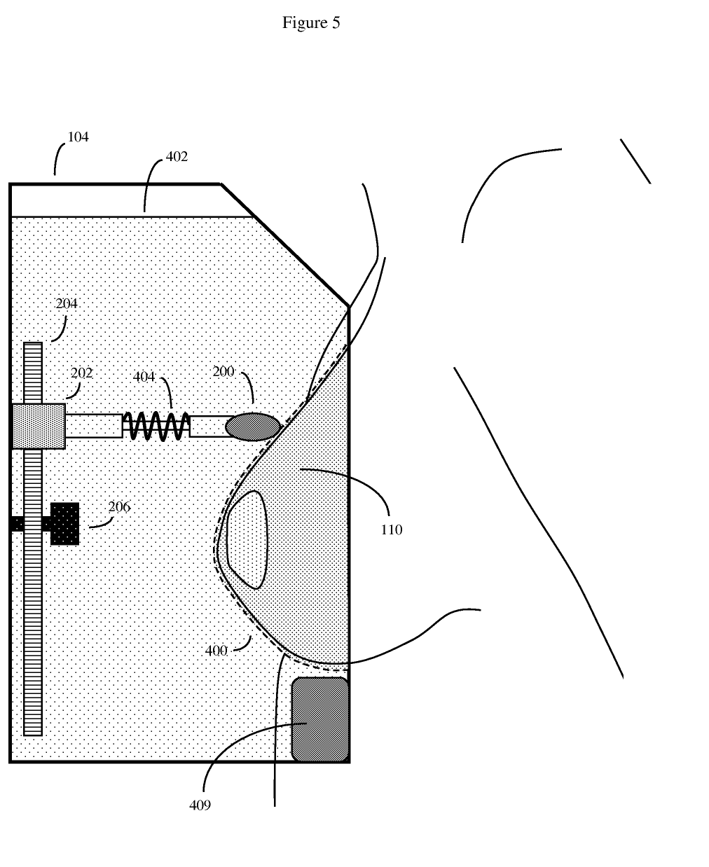

FIG. 5 shows an alternate embodiment of the device, more closely resembling a traditional mammography scanner, in which the patient is using the device standing up. Here the moveable ultrasonic transducer guidance device (104) is configured with the flexible membrane (400) on the side of the device, and the mechanism (204), (202) to drive the ultrasonic transducer (200) is mounted at a different up and down angle from the earlier embodiment. An optional platform (409) inside the device may help the user position the breast for optimal scanning. Otherwise most other components of the moveable ultrasonic transducer guidance device are otherwise the same or similar to the patient laying down version previously shown in FIGS. 1 and 2.

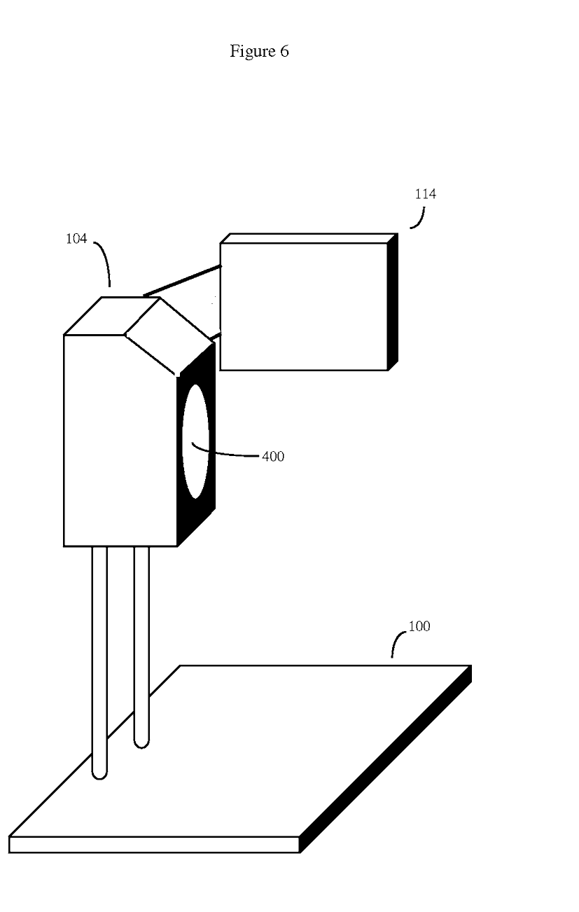

FIG. 6 shows an alternate embodiment of the ultrasonic breast examination system with the moveable ultrasonic transducer guidance device (114) positioned to be adjusted up and down in height relative to the base (110) to match various patient breast heights. In this configuration, the patient is standing up. Otherwise other aspects of the system, including the display device (114) are the same or similar as previously discussed for the patient laying down version previously discussed in FIGS. 1 and 2.

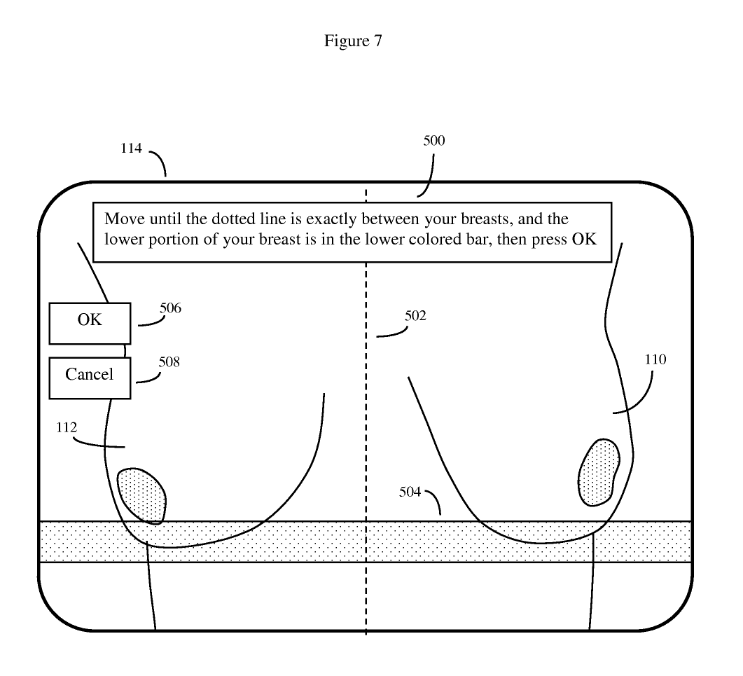

FIG. 7 shows an example of an image of the patient's breasts (110), (112) as displayed on the display device (114) or (116), showing how the system processor and software can display commands to direct the patient to manually reposition her body on the patient positioning platform. In FIG. 7, the image of both breasts may be provided by the patient positioning image sensor (106) when the moveable ultrasonic transducer guidance device (104) is in the “up” position, such as the configuration shown in FIG. 1. The system processor (often one or more microprocessors) and software may, for example, use take the image from image sensor (106), superimpose directions and reference marks onto the image, and display this image on the display device (114) or (116). If the display device (114) (116) is a touch sensitive display device, then the system processor and software may additionally indicate regions of the display device screen where the user may enter commands.

In the example shown in FIG. 7, the system is directing the patient (108), who may be using the system otherwise unassisted by local support personnel, to reposition herself on the patient positioning platform (110) until she is in the correct position to begin a scan. To do this, the system is displaying instructions (500), reference marks (502), (504), and touch sensitive buttons (506) (508) that can be used to control the system.

Thus in this example, the patient may be instructed in display screen writing (and optionally by computer generated speech as well) to “Move until the dotted line is exactly between your breasts, and the lower portion of your breast is in the lower colored bar, then press OK”. The patient will move until her breasts are midway between reference mark (502) and the lower portion of her breasts are in the reference mark zone (504). When the patient sees that she is in correct position, she may inform the system to proceed by pressing the “OK” screen “button” (506) or alternately canceling or returning to an earlier menu by pressing the “Cancel” screen “button” (508). Alternatively these steps may be done by a non-patient local operator of the system, or alternatively by a remote operator of the system if the system is connected to a network, such as the Internet.

It is also useful to build a video camera into the system simply to record the overall appearance of the patient's breasts for both record keeping and remote operator use, because some breast diseases can be seen by optical methods alone. For example, the camera may be useful for confirming inverted nipples, moles, obvious lumps, or cases where breast cancer may actually be spreading to the surface of the breast. Thus such an overall view video camera or patient positioning sensor (106) may be useful for standing up embodiments of the system as well.

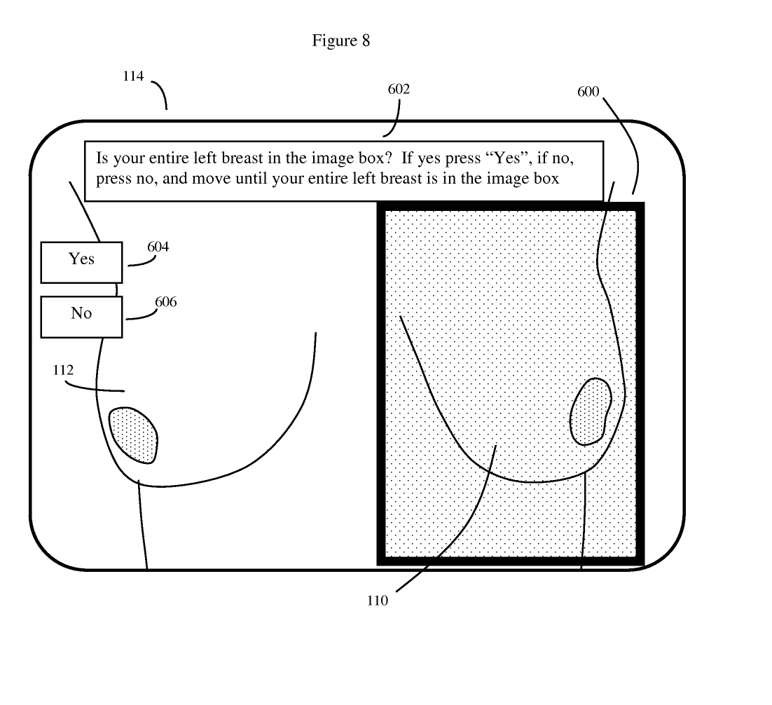

FIG. 8 shows an example of how a breast positioning image sensor (206) inside the moveable ultrasonic transducer guidance device (104) may look at the patient's breast through a transparent flexible side or bottom (400) once the ultrasonic transducer guidance device is positioned proximate to the patient's breast. Thus once the ultrasonic transducer guidance device (104) is positioned proximate to the patient's breast (110), images of the patient's breast are produced which may be also displayed on the display device (114), (116). These images may be used to insure that the patient (108) and the patient's breast (110) are properly positioned prior to scanning. This technique may also be used to help control the breast scanning process.

In this example, the system may prompt the patient or operator to view the image produced by the breast positioning image sensor (206), here shown inside box (600), and ask the patient either in writing (e.g. displayed on display device (114), (116), and/or orally by speech synthesizer) a question such as “Is your entire left breast in the image box? If yes press “Yes”, if no, press no, and move until your entire left breast is in the image box” (602). If the position of the breast (110) inside image box (602) appears adequate, then the patient or operator may inform the system to proceed by pressing a “Yes” button (604). If the position of the breast does not appear adequate, the patient or operator may inform the system of the need for further readjustments by pressing the “No” button (606).

In the example shown in FIG. 8, the image produced by a camera (206) inside the ultrasonic transducer guidance device is shown superimposed on an earlier image showing both the patient's two breasts, but alternatively only the image produced by camera (206) alone may be shown.

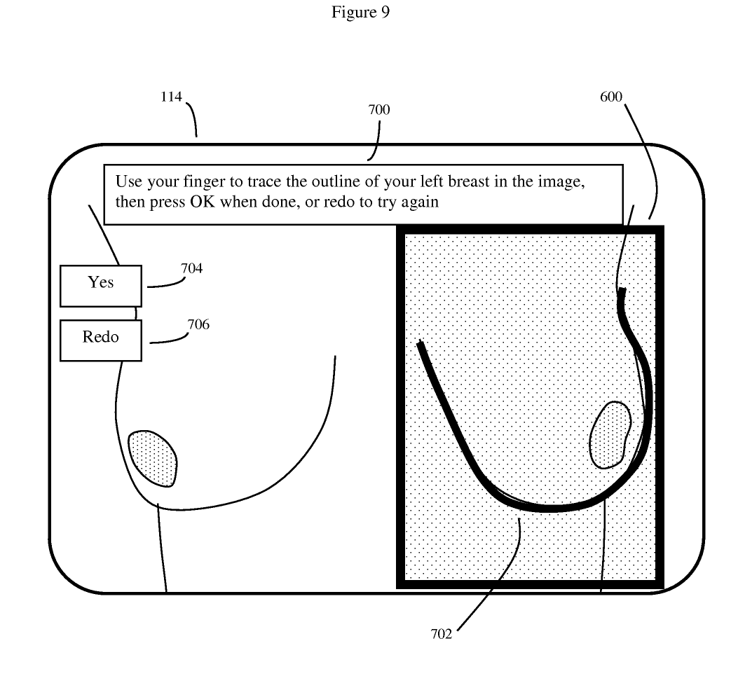

FIG. 9 shows an example of how either a patient or other user of the system may interact with a touch screen display device (114), (116) and, for example, inform the system as to the position of the boundaries of the breast (110), (112) as it is positioned underneath the ultrasonic transducer guidance device (104). Alternatively the positions of the breast boundaries can be determined by artificial vision systems or other user input modalities.

In this example, the patient or other system operator is informing the system of the boundaries of the breast using a touch screen display device such as (114), or (116). Other input methods may also be used, however. Here the system is again showing a detail of the breast (110) as visualized by breast positioning image sensor (206), and this is again shown inside box (600). Here the system is prompting the user to “Use your finger to trace the outline of your left breast in the image, then press OK when done, or redo to try again” (700). Here the patient or operator has traced the outline of the breast using a finger to press on the touch sensitive screen, and this outline is shown as (702). The patient or operator can then inform the system when the outline is acceptable by pressing the “Yes” button (704), or alternatively redo the outline by pressing the “Redo’ button (706).

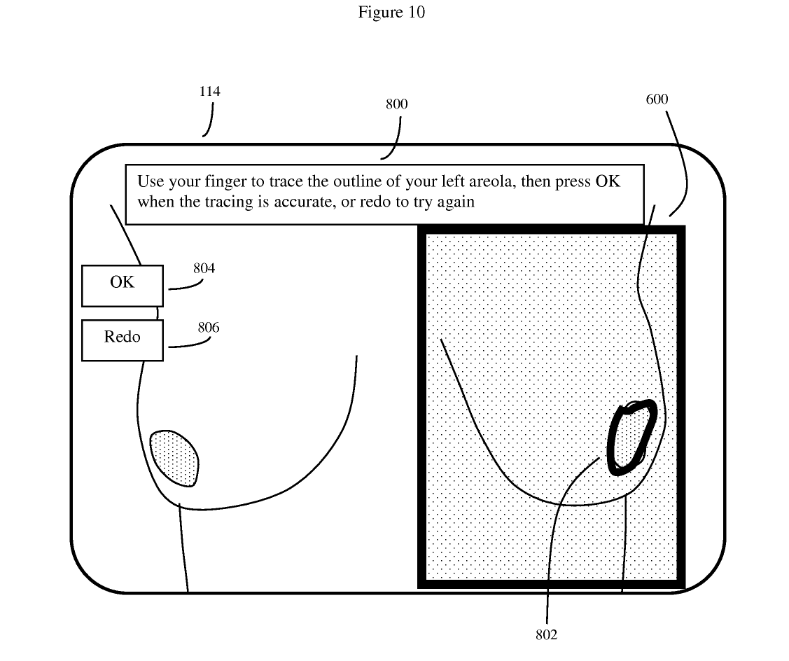

FIG. 10 shows an example of how either a patient or other system operator may interact with a touch screen display device (114) and, for example inform the system as to the position of the areola region of the breast as it is positioned underneath the ultrasonic transducer guidance device (104). Here the system is again showing a detail of the breast (110) as visualized by breast positioning image sensor (206), and this is again shown inside box (600). Alternatively positions of the breast areola or other breast structures can be determined by artificial vision systems or other user input modalities.

Here the system is prompting the user to “Use your finger to trace the outline of your left areola, then press OK when the tracing is accurate, or redo to try again” (800). Here the patient or operator has traced the outline of the breast areola using a finger to press on the touch sensitive screen, and this outline is shown as (802). The patient or operator can then inform the system when the outline is acceptable by pressing the “Yes” button (804), or alternatively redo the outline by pressing the “Redo” button (806).

This breast position information can be used both to control the scanning device itself, as well as provide useful annotation to the subsequent ultrasound breast images that are produced. Once the outlines of the breast and areola are determined, for example, then the breast image may be divided into quadrants and the locations of any structures or pathologies of interest can be referenced relative to these quadrants and breast coordinates.

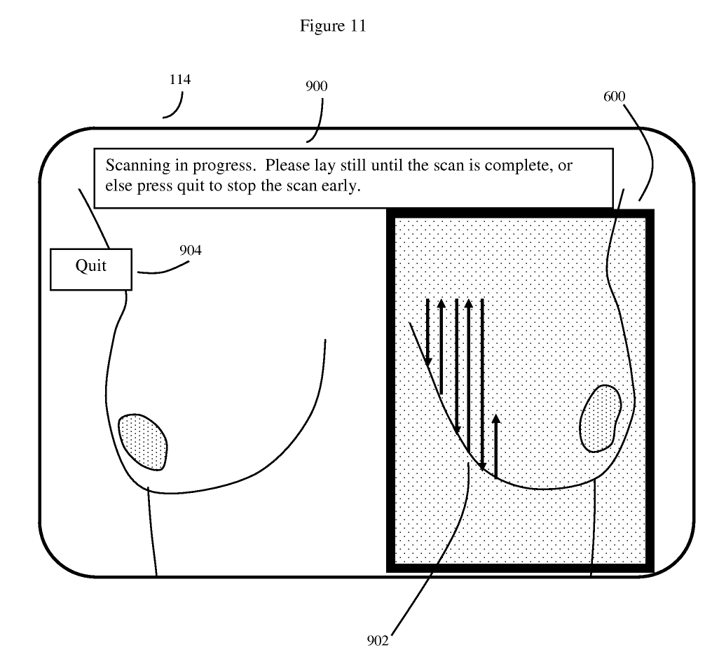

FIG. 11 shows an example of the display device, which again may be a touch screen display device (114), may look like during a breast scan process. This type of interface allows the patient to see the progress of the scan, and may also allow the patient to abort the scan early if the patient feels uncomfortable. In this example, the display (114) has informed the patient that the scan is operating by showing the message: “Scanning in progress. Please lay still until the scan is complete, or else press quit to stop the scan early” (900). A detail of the breast (110) as visualized by breast positioning image sensor (206), is shown inside box (600).

Here the system may simply show the actual movement of the ultrasonic transducer (200) and associated transport mechanisms (202), (204) as visualized by the breast positioning image sensor (206). In this embodiment, the system will thus show a video, somewhat resembling FIG. 3, which shows the actual ultrasonic transducer as it moves about the breast.

Alternatively the system may show a more stylized, computer generated image by, for example, first making a still photo of breast (110) before the scan commences, and then simply showing the movement of the tip of the ultrasonic transducer on the breast by a computer graphic overlay without showing the actual image of the transducer. This would produce a simpler image that might be less distracting to the patient. An example of such an abstracted or simplified scan pattern is shown in (902). Alternatively the progress of the scan can be shown by other graphical methods, such as a progress bar, a percent done number, or other method.

If the patient becomes uncomfortable or otherwise wishes to abort the scan early, the patient can terminate the scan by, for example, pressing a “Quit” button (904).

In this example, the image produced by camera (206) is shown superimposed onto a photo of both the patient's breasts, but of course this need not be done, and the image produced by camera or video device (206) may instead be shown by itself.

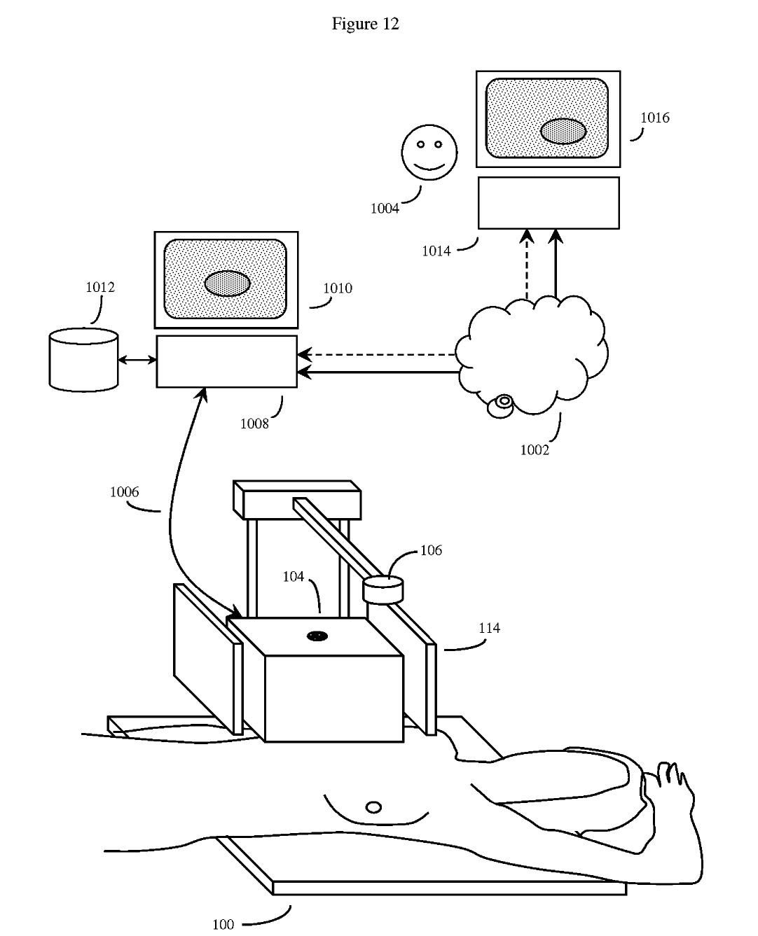

FIG. 12 shows an example of the ultrasonic breast examination system (100), (104), (106), (114) being controlled over a network, such as the internet (1002), by a remote operator (1004). In this example, the laying down version of the device is shown. In this example, the moveable ultrasonic transducer guidance device (104), as well as the patient positioning image sensor (106), the display devices (114), (116) and other electronic components may be connected by a wireless or electrical data transport cable (1006) to electronics (1008) that may, for example, contain both the processor and software that controls the automated medical ultrasonic transducer guidance device (104) as well as the electronics that drives the ultrasonic transducer head (200), and interprets the ultrasonic signal and produces an image on local display (1010). The local electronics may, for example, have one or more network interfaces to networks such as cell phone networks or the internet. Additionally, the local electronics may be connected to various mechanisms for local storage and print-out of the ultrasonic results, such as a printer, disk drive, or other data storage mechanism (1012).

The ultrasonic breast examination system may be controlled by more than one mechanism. In some embodiments, the local electronics (1008) may take charge of controlling the moveable ultrasonic transducer guidance device (104) that in turn controls the movement of the ultrasonic transducer (200). In this case the remote operator (1004) may either passively observe the images as reported over the network (1002), or may even not be involved at all. Here, for example, network (1002) may not even be used, and the results simply stored in local storage (1012) for subsequent analysis by a more skilled operator at a later time. Alternatively, remote operator (1004) may, for example, take command of the ultrasonic transducer guidance device (104) and use the remote electronics (1014) to send commands over the network to adjust the local electronics (1008). These commands may, for example, control the setting of the ultrasonic transducer or remotely control the scanning pattern of the ultrasonic transducer (200). The remote operator (1004) may also observe the ultrasonic scan through the remote video display (1016).

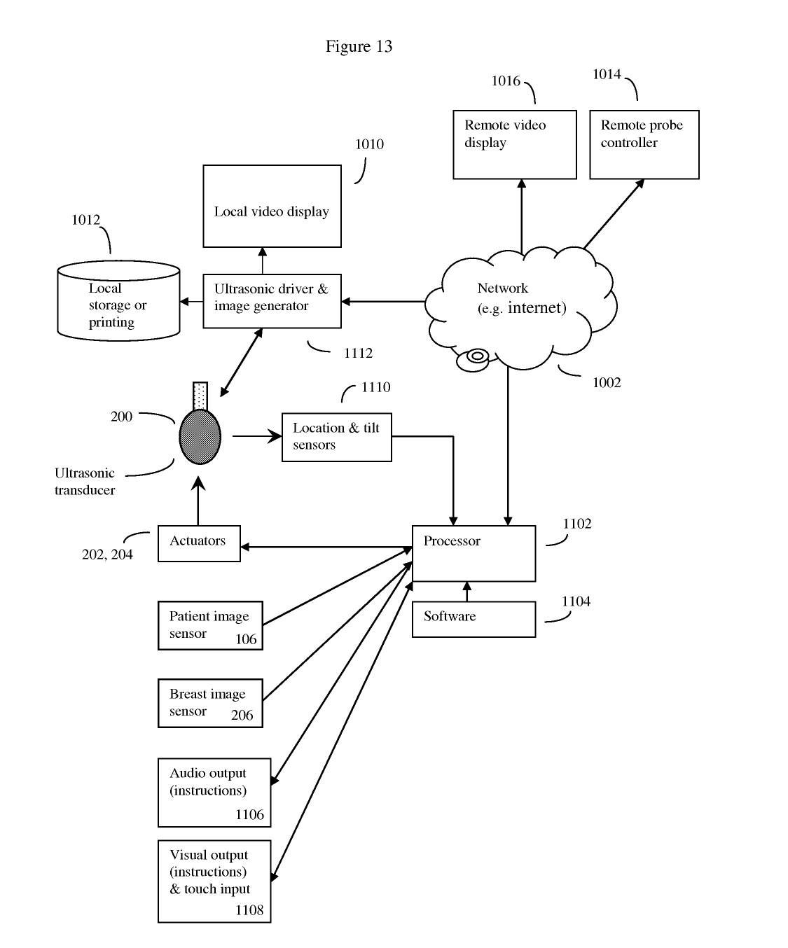

FIG. 13 shows an example of the electronics and software used in the system. Here the ultrasonic transducer (200) inside ultrasonic transducer guidance device (104) may have its position controlled by one or more actuators attached to positioning mechanisms (202) and (204).

The system will normally be controlled by at least one processor, such as a microprocessor (1102) running under the control of software (1104). In addition to running any actuators or positioning mechanisms (202), (204), the processor may also process the video input from least one patient positioning image sensor (106) and/or breast positioning image sensor (206), send audio commands (1106) to the patient or other local operators instructing then as to how to use the system. The processor (1102) and software (1104) may also send images and visual commands to the patient or local operators, and receive input from the patient or local operators (1108), often using touch sensitive display devices (114) or (116).

In some versions, normal off-the shelf ultrasonic equipment may be retrofitted onto the transducer guiding system. In this case, the ultrasonic driver and image generator (1112) may or may not be integrated in or be under the control of processor and software (1102) (1104). However for greater control, normally putting the ultrasonic driver and image generator (1112) under control of processor (1102) will generally be desirable.

As previously discussed, both processor (1102) and the ultrasonic driver and image generator (1112) will often have network connections allowing them to exchange data, and optionally be controlled by, remote networked equipment over a network such as the internet (1002). In this case, the remote networked equipment may include a remote video display (1016) and an optional electronics or software to control the moveable ultrasonic transducer guidance device (104) and/or the ultrasonic driver and image generator (1112), and other system electronics.

This connection to the internet can be a direct electrical wire connection, or it can be a wireless connection, and here the term electronically connected to the Internet will be defined to encompass both methods.

1. A medical ultrasonic examination system for a patient, comprising:
a patient positioning platform, and a moveable ultrasonic transducer guidance device;
said moveable ultrasonic transducer guidance device comprising a container with a flexible side or bottom, a moveable ultrasonic transducer configured to move within said container, and a mechanism to automatically move said moveable ultrasonic transducer;
said flexible side or bottom capable of conforming to the shape of at least one portion of said patient when said transducer guidance device is positioned about said at least one portion of said patient;
said moveable ultrasonic transducer guidance device being capable of moving from at least a first position away from said at least one portion of said patient to a second position proximate said at least one portion of said patient;
a processor and software configured to direct said mechanism to automatically move said moveable transducer in a scanning pattern intended to enable echo information obtained by said ultrasonic transducer to be assembled into at least one image representing the interior of said portion of said patient.

2. The system of claim 1, in which the portion of said patient is the breast.

3. The system of claim 2, in which said container is filled with fluid, and the pressure of said fluid pressing against said flexible side or bottom causes said flexible side or bottom to tightly adhere to at least one breast of said patient when said transducer guidance device is positioned about said at least one breast of said patient.

4. The system of claim 2, in which the flexible side or bottom is a transparent flexible side or bottom, and said moveable ultrasonic transducer guidance device additionally comprises at least one breast positioning image sensor configured to view a breast positioned against said transparent flexible side or bottom, and said system additionally comprises least one display device configured to display at least some of the images from said breast positioning image sensor.

5. The system of claim 4, in which said at least one display device is a touch screen display device configured to receive touches conveying information pertaining to the location of at least one patient breast structure that is displayed on said display device from either said patient or from another operator of said system.

6. The system of claim 4, in which said software comprises artificial vision software configured to automatically determine the location of at least one patient breast structure.

7. The system of claim 2, wherein said processor and software issue commands to direct a patient to manually reposition her body while using said system.

8. The system of claim 2, wherein said system is electronically connected to a network, and in which said at least one image representing the interior of said patient's breast may be viewed by a remote user, who may be a skilled user.

9. The system of claim 2, wherein said system is electronically connected to a network, and wherein a remote user, who may be a skilled user, interacts with said at least one processor and software to direct the position of said ultrasonic transducer.

10. The system of claim 2, wherein said patient positioning platform, said moveable ultrasonic transducer guidance device; and at least one display device are mechanically coupled together to form a single unit; and
said moveable ultrasonic transducer guidance device is configured to scan only single patient breast, and said patient positioning platform is mechanically configured so as to allow a patient to first scan one breast, and then reposition her body to then scan the other breast.

11. A medical ultrasonic breast examination system for a patient, comprising:
a patient positioning platform with a moveable ultrasonic transducer guidance device;
said moveable ultrasonic transducer guidance device comprising a container with a transparent flexible side or bottom, at least one breast positioning image sensor configured to view a breast positioned against said transparent flexible side or bottom, a moveable ultrasonic transducer configured to move within said container, and a mechanism to automatically move said moveable ultrasonic transducer;
said transparent flexible side or bottom capable of conforming to the shape of at least one breast of said patient when said transducer guidance device is positioned about said at least one breast of said patient;
in which said container is filled with fluid, and the pressure of said fluid pressing against said transparent flexible side or bottom causes said transparent flexible side or bottom to tightly adhere to at least one breast of said patient when said transducer guidance device is positioned about said at least one breast of said patient;
said moveable ultrasonic transducer guidance device being capable of moving from at least a first position away from said at least one breast of said patient to a second position proximate said at least one breast of said patient;
at least one display device configured to display at least some of the images from said breast positioning image sensor;
a processor and software configured to direct said mechanism to automatically move said moveable transducer in a scanning pattern intended to enable echo information obtained by said ultrasonic transducer to be assembled into at least one image representing the interior of said patient's breast;
wherein said patient positioning platform, said moveable ultrasonic transducer guidance device; and said at least one display device are mechanically coupled together to form a single unit.

12. The system of claim 11, in which said at least one display device is a touch screen display device configured to receive touches conveying information pertaining to the location of at least one patient breast structure that is displayed on said display device from either said patient or from another operator of said system.

13. The system of claim 11, in which said software comprises artificial vision software configured to automatically determine the location of at least one patient breast structure.

14. The system of claim 11, wherein said processor and software issue commands to direct a patient to manually reposition her body while using said system.

15. The system of claim 11, wherein said system is electronically connected to a network, and in which said at least one image representing the interior of said patient's breast may be viewed by a remote user, who may be a skilled user.

16. The system of claim 11, wherein said system is electronically connected to a network, and wherein a remote user, who may be a skilled user, interacts with said at least one processor and software to direct the position of said ultrasonic transducer.

17. The system of claim 11, wherein said moveable ultrasonic transducer guidance device is configured to scan only single patient breast, and said patient positioning platform and said at least one display device are mechanically configured so as to allow a patient to first scan one breast, and then reposition her body to then scan the other breast.

18. A method for automatically performing a medical ultrasonic breast exam of a patient, said method comprising;
obtaining a patient positioning platform with at least one patient positioning image sensor and, a moveable ultrasonic transducer guidance device;
said moveable ultrasonic transducer guidance device comprising a fluid filled container with a flexible side or bottom, a moveable ultrasonic transducer configured to move within said container, and a mechanism to automatically move said moveable ultrasonic transducer about at least one breast of said patient;
positioning said moveable ultrasonic transducer guidance device that said flexible side or bottom conforms to the shape of said at least one breast of said patient, and the pressure of said fluid on said flexible side or bottom causes said flexible side or bottom to tightly adhere to said at least one breast of said patient;
using a processor and software to direct said mechanism to automatically move said moveable ultrasonic transducer about said at least one breast of said patient, thereby obtaining ultrasonic image data;
and assembling said ultrasonic image data into at least one image representing the interior of said patient's breast.

19. The method of claim 18, further displaying an image of at least one breast of said patient on a touch screen display device;
receiving touch information from said touch screen display device pertaining to the location of at least one breast structure;
and using this touch information to either inform said processor and software of how best to automatically move said moveable ultrasonic transducer about said at least one breast or said patient; or
control the appearance of said at least one image representing the interior of said patient's breast.

20. The method of claim 18, further connecting said patient positioning platform, said moveable ultrasonic transducer guidance device, said moveable ultrasonic transducer, said processor and said software to a network; and
transmitting said at least one image representing the interior of said patient's breast over said network; or
allowing a remote user, who may be a skilled user, interact with said at least one processor and software to direct the position of said ultrasonic transducer.
